(19) United States
(12) Patent Application Publication
(10) Pub. No.: US 2007/0191438 A1
(43) Pub. Date: Aug. 16, 2007

(54) Methods for the treatment of hypertension

(76) Inventor: Susan P. Rohrer, Scotch Plains, NJ (US)

Correspondence Address: MERCK AND CO., INC, P O BOX 2000, RAHWAY, NJ, 07065-0907, US

(21) Appl. No.: 10/594,372

(22) PCT Filed: Apr. 1, 2005
(86) PCT No.: PCT/US05/11186

§ 371 Date: Sep. 27, 2006

Related U.S. Application Data
(60) Provisional application No. 60/559,985, filed on Apr. 6, 2004.

(51) Int. Cl. A61K 31/4192; A61K 31/554; A61K 31/549; A61K 31/4188; A61K 31/416; A61K 31/455; A61K 31/138

(52) U.S. Cl. 514/359; 514/393; 514/406; 514/394; 514/381; 514/223.5; 514/651; 514/355; 514/211.07

(57) ABSTRACT

This invention relates to the treatment of hypertension, cardiac dysfunction or stroke by the administration of an estrogen receptor beta (ER&bgr;) selective agonist either as a single agent, or in combination with other agents.

### BACKGROUND OF THE INVENTION

This invention relates to the treatment of hypertension, cardiac dysfunction or stroke by the administration of an estrogen receptor beta (ERβ) selective agonist either as a single agent, or in combination with other agents.

Hypertension affects 1 in 4 American adults. Hypertension can damage the arteries, heart, and kidneys, and lead to atherosclerosis and stroke. Hypertension treatment generally depends on the severity of the disease, in addition to other health problems, such as heart failure, diabetes, or pregnancy. Such treatments can involve lifestyle changes, medication or a combination of both. Treatment of hypertension decreases the risk of heart failure, coronary artery disease, heart attack, abnormal heartbeats, stroke, and kidney disease, and reduces the risk of death from these conditions.

Cardiac dysfunction, which includes enlarged hearts, increased heart rate, decreased cardiac output, and variable left ventricular systolic blood pressure, has been described in mice lacking the gene for tryptophan hydroxylase, the rate limiting enzyme involved in serotonin synthesis (Cote, Thevenot, Fligny, Fromes, Darmon, Ripoche, Bayard, Hanoun, Saurini, Lechat, Dandolo, Hamon, Mallet, Vodjdani (2003) PNAS 100: 13525-13530). These alterations in cardiac function lead progressively to heart failure.

Stroke is a type of cardiovascular disease that affects the arteries leading to and within the brain. A stroke occurs when a blood vessel that carries oxygen and nutrients to the brain is either blocked by a clot or bursts. Clots that block an artery cause ischemic strokes. This is the most common type of stroke, accounting for 70-80 percent of all strokes. Ruptured blood vessels cause hemorrhagic or bleeding strokes. When part of the brain dies from lack of blood flow, the part of the body it controls is affected. Strokes can cause paralysis, affect language and vision, and cause other problems.

A role for ER-beta in hypertension has been suggested by studies conducted in ER-beta knockout mice (Zhu, Bian, Lu, Karas, Bao, Cox, Hodgin, Shaul, Thoren, Smithies, Gustafsson, Mendelsohn (2002) Science 295: 505-508). These mice display increased systolic blood pressure as they age suggesting that ER-beta's presence is required for the maintenance of normal blood pressure. Interestingly, human mutations in ER-beta have been shown to be associated with the development of hypertension at menopause (Ogawa, Emi, Shiraki, Hosoi, Ouchi, and Inoue (2000) J. Hum. Genet. 45: 327-330).

### SUMMARY OF THE INVENTION

The present invention relates a method of treating hypertension, cardiac dysfunction or stroke with an ERβ agonist. The present invention also relates to the use of an ERβ agonist for the preparation of a medicament useful in the treatment of hypertension, cardiac dysfunction or stroke. The ERβ agonist can be administered alone or in combination with another anti-hypertensive agent.

### DETAILED DESCRIPTION OF THE INVENTION

The present invention relates a method of treating hypertension, cardiac dysfunction or stroke with an ERβ agonist. The ERβ agonist can be administered alone or in combination with another anti-hypertensive agent.

In an embodiment of the invention, the ERβ agonist exhibits binding affinities to the estrogen receptor β-subtype in the range of an IC₅₀ of about 0.6 nm to about 126 nm.

Non-limiting examples of ERβ selective agonists include compounds described in International Publication WO 02/41835 of the formula: 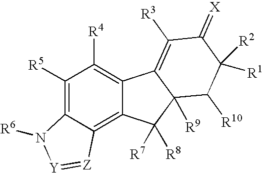
wherein X is 0 or N—OR^{a};
Y is N or CH;
Z is N or CR^{f};
R¹ is hydrogen or C₁₋₆alkyl;
R² is hydrogen, hydroxy, iodo or C₁₋₆alkyl;
R³ is hydrogen, fluoro, chloro, bromo, iodo, cyano, nitro, NR^{a}R^{c}, OR^{a}, S(O)R^{a}, SO₂R^{a}, SR^{a}, C(═O)R^{a}, CO₂R^{c}, CONR^{a}R^{c}, C₁₋₁₀alkyl, C₂₋₁₀alkenyl, C₂₋₁₀alkynyl, C₃₋₇cycloalkyl, 4-7 membered heterocycloalkyl, cycloalkylalkyl, aryl, heteroaryl, arylalkyl, or heteroarylalkyl, wherein said alkyl, alkenyl, alkynyl, cycloalkyl, aryl and heteroaryl groups are optionally substituted with 1, 2 or 3 groups selected from the group consisting of fluoro, chloro, bromo, iodo, cyano, OR^{a}, NR^{a}R^{c}, O(C═O)R^{a}, O(C═O)NR^{a}R^{c}, NR^{a}(C═O)R^{c}, NR^{a}(C═O)OR^{c}, C(═O)R^{a}, CO₂R^{a}, CONR^{a}R^{c}, CSNR^{a}R^{c}, SR^{a}, S(O)R^{a}, SO₂R^{a}, SO₂NR^{a}R^{c}, LR^{d}, and MLR^{d};
R⁴ is hydrogen, hydroxy, methyl, fluoro or chloro;
R⁵ is hydrogen, hydroxy, fluoro or chloro;
R⁶ is hydrogen, (C═O)R^{a} or (C═O)OR^{a};
R⁷ is hydrogen, fluoro, chloro or C₁₋₆alkyl;
R⁸ is hydrogen, fluoro, chloro or C₁₋₆alkyl;
   or R⁷ and R⁸, when taken together with the carbon atom to which they are attached, form a carbonyl group;
R⁹ is hydrogen, C₁₋₁₀alkyl, C₂₋₁₀alkenyl, C₃₋₆cycloalkyl, C₃₋₆cycloalkylalkyl, aryl, heteroaryl, arylalkyl or heteroarylalkyl, wherein said alkyl, alkenyl, cycloalkyl, cycloalkylalkyl, aryl, heteroaryl, arylalkyl and heteroarylalkyl groups are optionally substituted with chloro, bromo, OR^{b}, SR^{b} or 1-5 fluoro;
   or R⁹ and R¹, when taken together with the three intervening carbon atoms to which they are attached, form a 5-6 membered cycloalkyl ring which is optionally substituted with 1-3 fluoro, chloro, C₁₋₆alkyl, C₂₋₆alkenyl or C₃₋₆cycloalkylalkyl, wherein said alkyl, alkenyl and cycloalkylalkyl, groups are optionally substituted with chloro, OR^{b}, SR^{b} or 1-5 fluoro;
R¹⁰ is hydrogen or C₁₋₁₀alkyl;
R^{a} is hydrogen, C₁₋₁₀alkyl or phenyl, wherein said alkyl group is optionally substituted with hydroxy, amino, O(C₁₋₄alkyl), NH(C₁₋₄alkyl), N(C₁₋₄alkyl)₂, phenyl or 1-5 fluoro, and wherein said phenyl group is optionally substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H and C(O)(C₁₋₄alkyl);
R^{b} is hydrogen, C₁₋₁₀alkyl, benzyl or phenyl, wherein said phenyl group is optionally substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H and C(O)(C₁₋₄alkyl);
R^{c} is hydrogen, C₁₋₁₀alkyl or phenyl, wherein said phenyl group is optionally substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H and C(O)(C₁₋₄alkyl);
   or R^{a} and R^{c}, whether or not on the same atom, can be taken together with any attached and intervening atoms to form a 4-7 membered ring;
R^{d} is NRbR^{c}, OR^{a}, CO₂R^{a}, O(C═O)R^{a}, CN, NR^{c}(C═O)R^{b}, CONR^{a}R^{c}, SO₂NR^{a}R^{c or a} 4-7 membered N-heterocycloalkyl ring that is optionally interrupted by O, S, NR^{c}, or C═O;
R^{e} is hydrogen, C₁₋₆alkyl, C₂₋₆alkenyl, CF₃, halo, O(C₁₋₄alkyl), NH₂, NH(C₁₋₄alkyl) or N(C₁₋₄alkyl)₂;
R^{f} is hydrogen, C₁₋₆alkyl, C₂₋₆alkenyl, CF₃, halo, O(C₁₋₄alkyl), NO₂, NH₂, NH(C₁₋₄alkyl) or N(C₁₋₄alkyl)₂;
L is CR^{b}R^{c}, C₂₋₆ alkylene or C₂₋₆ alkenylene, wherein said alkylene and alkenylene groups are optionally interrupted by O, S, or NR^{c};
M is O, S, NR^{c}, C═O, O(C═O), (C═O)O, NR^{c}(C═O) or (C═O)NR^{c};
or a salt or stereoisomer thereof.

In a class of the invention, X is O, N—OH or N—OCH₃. In a subclass of the invention, X is O.

In a class of the invention, Y is N or CH.

In a class of the invention, Z is N, CH, CF or CCl. In a subclass of the invention Z is N or CH.

In a class of the invention, R¹ is hydrogen or C₁₋₃alkyl.

In a class of the invention, R² is hydrogen, hydroxy, iodo or C₁₋₃alkyl.

In a class of the invention, R³ is hydrogen, chloro, bromo, iodo, C₁₋₁₀alkyl, C₂₋₁₀alkenyl, C₃₋₇cycloalkyl or aryl, wherein said alkyl, alkenyl, cycloalkyl and aryl groups are optionally substituted with 1, 2 or 3 groups selected from the group consisting of fluoro, OR^{a}, NR^{a}R^{c}, LR^{d} and MLR^{d}.

In a class of the invention, R⁴ is hydrogen, methyl or fluoro.

In a class of the invention, R⁵ is hydrogen or fluoro.

In a class of the invention, R⁶ is hydrogen or C(═O)OR^{a}.

In a class of the invention, R⁷ is hydrogen or C₁₋₆alkyl.

In a class of the invention, R⁸ is hydrogen or C₁₋₆alkyl. In a class of the invention, R⁹ is C₁₋₁₀alkyl, C₂₋₁₀alkenyl, C₃₋₆cycloalkyl or cycloalkylalkyl.

In a class of the invention, R¹⁰ is hydrogen.

Specific compounds include, but are not limited to:
9a-ethyl-1,6-dimethyl-8,9,9a,10-tetrahydroindeno[2,1-e]indol-7(3H)-one;
9a-ethyl-6-methyl-8,9,9a,10-tetrahydroindeno[2,1-e]indol-7(3H)-one;
1-chloro-9a-ethyl-6-methyl-8,9,9a,10-tetrahydroindeno[2,1-e]indol-7(3H)-one;
9a-ethyl-6-methyl-1-nitro-8,9,9a,10-tetrahydroindeno[2,1-e]indol-7(3H)-one;
6-acetyl-9a-butyl-4-fluoro-8,9,9a,10-tetrahydroindeno[2,1-e]indol-7(3H)-one;
6-methyl-9a-propyl-8,9,9a,10-tetrahydroindeno[2,1-e]indol-7(3H)-one;
9a-ethyl-4-fluoro-6-methyl-8,9,9a,10-tetrahydroindeno[2,1-e]indol-7(3H)-one;
6,9a-diethyl-4-fluoro-8,9,9a,10-tetrahydroindeno[2,1-e]indol-7(3H)-one;
9a-butyl-4-fluoro-6-methyl-8,9,9a,10-tetrahydroindeno[2,1-e]indol-7(3H)-one;
9a-butyl-6-ethyl-4-fluoro-8,9,9a,10-tetrahydroindeno[2,1-e]indol-7(3H)-one;
6,9a-dimethyl-8,9,9a,10-tetrahydroindeno[2,1-e]indazol-7(3H)-one;
6-bromo-9a-methyl-8,9,9a,10-tetrahydroindeno[2,1-e]indazol-7(3H)-one;
9a-ethyl-8,9,9a,10-tetrahydroindeno[2,1-e]indazol-7(3H)-one;
9a-ethyl-6-methyl-8,9,9a,10-tetrahydroindeno[2,1-e]indazol-7(3H)-one;
6-bromo-9a-ethyl-8,9,9a,10-tetrahydroindeno[2,1-e]indazol-7(3H)-one;
9a-ethyl-6-trifluoromethyl-8,9,9a,10-tetrahydroindeno[2,1-e]indazol-7(3H)-one;
9a-ethyl-6-{4-[2-(1-piperidinyl)ethoxy]phenyl }-8,9,9a,10-tetrahydroindeno[2,1-e]indazol-7(3H)-one hydrochloride salt;
9a-ethyl-6-(4-hydroxyphenyl)-8,9,9a,10-tetrahydroindeno[2,1-e]indazol-7(3H)-one;
9a-ethyl-6-vinyl-8,9,9a,10-tetrahydroindeno[2,1-e]indazol-7(3H)-one;
6,9a-diethyl-8,9,9a,10-tetrahydroindeno[2,1-e]indazol-7(3H)-one;
6-allyl-9a-ethyl-8,9,9a,10-tetrahydroindeno[2,1-e]indazol-7(3H)-one;
9a-ethyl-6-isopropyl-8,9,9a,10-tetrahydroindeno[2,1-e]indazol-7(3H)-one;
6-butyl-9a-ethyl-8,9,9a,10-tetrahydroindeno[2,1-e]indazol-7(3H)-one;
6-cyclopentyl-9a-ethyl-8,9,9a,10-tetrahydroindeno[2,1-e]indazol-7(3H)-one;
6-cyano-9a-ethyl-8,9,9a,10-tetrahydroindeno[2,1-e]indazol-7(3H)-one;
9a-ethyl-6-methoxy-8,9,9a,10-tetrahydroindeno[2,1-e]indazol-7(3H)-one;
1-chloro-9a-ethyl-6-methyl-8,9,9a,10-tetrahydroindeno[2,1-e]indazol-7(3H)-one;
1-bromo-9a-ethyl-6-methyl-8,9,9a,10-tetrahydroindeno[2,1-e]indazol-7(3H)-one;
9a-ethyl-6-methyl-9,9a-dihydroindeno[2,1-e]indazole-7,10(3H,8H)-dione;
10-chloro-9a-ethyl-6-methyl-8,9,9a,10-tetrahydroindeno[2,1-e]indazol-7(3H)-one;
10-azido-9a-ethyl-6-methyl-8,9,9a,10-tetrahydroindeno[2,1-e]indazol-7(3H)-one;
6-bromo-9a-ethyl-9,9a-dihydroindeno[2,1-e]indazole-7,10(3H,8H)-dione;
10-amino-9a-ethyl-6-methyl-8,9,9a,10-tetrahydroindeno[2,1-e]indazol-7(3H)-one;
9a-ethyl-10-methoxy-6-methyl-8,9,9a,10-tetrahydroindeno[2,1-e]indazol-7(3H)-one;
9a-ethyl-6,10-dimethyl-8,9,9a,10-tetrahydroindeno[2,1-e]indazol-7(3H)-one;
9a-ethyl-4-fluoro-6-methyl-8,9,9a,10-tetrahydroindeno[2,1-e]indazol-7(3H)-one;
6,9a-diethyl-4-fluoro-8,9,9a,10-tetrahydroindeno[2,1-e]indazol-7(3H)-one;
6-bromo-9a-ethyl-4-fluoro-8,9,9a,10-tetrahydroindeno[2,1-e]indazol-7(3H)-one;
9a-ethyl4-fluoro-6-trifluoroinethyl-8,9,9a,10-tetrahydroindeno[2,1-e]indazol-7(3H)-one;
6-methyl-9a-propyl-8,9,9a,10-tetrahydroindeno[2,1-e]indazol-7(3H)-one;
6-bromo-9a-propyl-8,9,9a,10-tetrahydroindeno[2,1-e]indazol-7(31H)-one;
6-cyano-9a-propyl-8,9,9a,10-tetrahydroindeno[2,1-e]indazol-7(3H)-one;
6-methyl-9a-propyl-8,9,9a,10-tetrahydroindeno[2,1-e]indazol-7(3H)-one oxime;
9a-butyl-8,9,9a,10-tetrahydroindeno[2,1-e]indazol-7(3H)-one;
6-bromo-9a-butyl-8,9,9a,10-tetrahydroindeno[2,1-e]indazol-7(3H)-one;
9a-butyl-6-trifluoromethyl-8,9,9a,10-tetrahydroindeno[2,1-e]indazol-7(3H)-one;
9a-butyl-6-metbyl-8,9,9a,10-tetrahydroindeno[2,1-e]indazol-7(3H)-one;
9a-butyl-6-ethyl-8,9,9a,10-tetrahydroindeno[2,1-e]indazol-7(3H)-one;
9a-(3,3-dimethylbutyl)-6-methyl-8,9,9a,10-tetrahydroindeno[2,1-e]indazol-7(3H)-one;
9a-butyl-6-ethyl-4-fluoro-8,9,9a,10-tetrahydroindeno[2,1-e]indazol-7(3H)-one;
6-acetyl-9a-butyl-4-fluoro-8,9,9a,10-tetrahydroindeno[2,1-e]indazol-7(3H)-one;
9a-butyl-4-fluoro-6-methyl-8,9,9a,10-tetrahydroindeno[2,1-e]indazol-7(311)-one;
6-bromo-9a-butyl-4-fluoro-8,9,9a,10-tetrahydroindeno[2,1-e]indazol-7(3H)-one;
9a-butyl-6-cyano-4-fluoro-8,9,9a,10-tetrahydroindeno[2,1-e]indazol-7(311)-one;
9a-butyl-4-fluoro-6-trifluoromethyl-8,9,9a,10-tetrahydroindeno[2,1-e]indazol-7(3H)-one;
6-methyl-3,9,10,11-tetrahydro-8,10a-methanoazuleno[2,1-e]indazol-7(8H)-one;
6-ethyl-3,9,10,11-tetrahydro-8,10a-methanoazuleno[2,1-e]indazol-7(8H)-one;
9a-ethyl-6-methyl-8,9,9a,10-tetrahydrofluoreno[1,2-d]imidazol-7(3H)-one;
6-bromo-9a-ethyl-8,9,9a,10-tetrahydrofluoreno[1,2-d]imidazol-7(3H1)-one;
6,9a-diethyl-4-fluoro-8,9,9a,10-tetrahydrofluoreno[1,2-d]imidazol-7(3H)-one;
9a-butyl-6-ethyl-4-fluoro-8,9,9a,10-tetrahydrofluoreno[1,2-d]imidazol-7(3H)-one;
9a-ethyl-8,9,9a,10-tetrahydrofluoreno[1,2-d][1,2,3]triazol-7(3H)-one;
9a-ethyl-6-methyl-8,9,9a,10-tetrahydrofluoreno[1,2-d][1,2,3]triazol-7(3H)-one;
6-allyl-9a-ethyl-8,9,9a,10-tetrahydrofluoreno[1,2-d][1,2,3]triazol-7(3H)-one;
9a-ethyl-6-propyl-8,9,9a,10-tetrahydrofluoreno[1,2-d][1,2,3]triazol-7(3H)-one;
9a-ethyl-6-trifluoromethyl-8,9,9a,10-tetrahydrofluoreno[1,2-d][1,2,3]triazol-7(3H)-one;
6-bromo-9a-etlyl-8,9,9a,10-tetrahydrofluoreno[1,2-d][1,2,3]triazol-7(3H)-one;
6,9a-diethyl-8,9,9a,10-tetrahydrofluoreno[1,2-d][1,2,3]triazol-7(3H)-one;
6-butyl-9a-ethyl-8,9,9a,10-tetrahydrofluoreno[1,2-d][1,2,3]triazol-7(3H)-one;
9a-ethyl-6-(4-hydroxyphenyl)-8,9,9a,10-tetrahydrofluoreno[1,2-d][1,2,3]triazol-7(3H)-one;
6-bromo-9a-propyl-8,9,9a,10-tetrahydrofluoreno[1,2-d][1,2,3]triazol-7(3H)-one;
6-methyl-9a-prapyl-8,9,9a,10-tetrahydrofluoreno[1,2-d][1,2,3]triazol-7(3H)-one;
9a-propyl-6-viayl-8,9,9a,10-tetrahydrofluoreno[1,2-d][1,2,3]triazol-7(3H)-one;
6-ethyl-9a-propyl-8,9,9a,10-tetrahydrofluoreno[1,2-d][1,2,3]triazol-7(3H)-one;
6-allyl-9a-propyl-8,9,9a,10-tetrahydrofluoreno[1,2-d][1,2,3]triazol-7(3H)-one;
6,9a-dipropyl-8,9,9a,10-tetrahydrofluoreno[1,2-d][1 ,2,3]triazol-7(3H)-one;
6-bromo-9a-butyl-8,9,9a,10-tetrahydrofluoreno[1,2-d][1,2,3]triazol-7(3H)-one;
9a-butyl-6-methyl-8,9,9a,10-tetrahydrofluoreno[1,2-d][1,2,3]triazol-7(3H)-one;
9a-butyl-6-ethyl-8,9,9a,10-tetrahydrofluoreno[1,2-d][1,2,3]triazol-7(3H)-one;
6-allyl-9a-butyl-8,9,9a,10-tetrahydrofluoreno[1,2-d][1,2,3]triazol-7(3H)-one;
9a-butyl-6-propyl-8,9,9a,10-tetrahydrofluoreno[ 1,2-d][1,2,3]triazol-7(3H)-one;
9a-butyl-6-trifluoromethyl-8,9,9a,10-tetrahydrofluoreno[ 1,2-d][1,2,3]triazol-7(3H)-one;
9a-butyl-6-(2-furyl)-8,9,9a,10-tetrahydrofluoreno[ 1,2-d][1,2,3]triazol-7(3H)-one;
6,9a-diethyl-4-fluoro-8,9,9a,10-tetrahydrofluoreno[ 1,2-d][1,2,3]triazol-7(3H)-one;
9a-butyl-6-ethyl-4-fluoro-8,9,9a,10-tetrahydrofluoreno[1,2-d][1,2,3]triazol-7(3B)-one;
   or a salt or stereoisomer thereof.

For use in medicine, the salts of the ERβ agonist compounds refer to non-toxic “pharmaceutically acceptable salts.” Other salts may, however, be useful in the preparation of the compounds according to the invention or of their pharmaceutically acceptable salts. When the compounds of the present invention contain a basic group, salts encompassed within the term “pharmaceutically acceptable salts” refer to non-toxic salts which are generally prepared by reacting the free base with a suitable organic or inorganic acid. Representative salts include the following: acetate, benzenesulfonate, benzoate, bicarbonate, bisulfate, bitartrate, borate, bromide, calcium, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The ERβ agonist compounds can have chiral centers and occur as racemates, racemic mixtures, diastereomeric mixtures, and as individual diastereomers, or enantiomers with all isomeric forms being included in the present invention. Therefore, where a compound is chiral, the separate enantiomers, substantially free of the other, are included within the scope; further included are all mixtures of the two enantiomers. Also included within the scope are polymorphs, hydrates and solvates of the compounds of the instant invention.

Also included are prodrugs of the ERβ agonist compounds. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

The term “alkyl” shall mean a substituting univalent group derived by conceptual removal of one hydrogen atom from a straight or branched-chain acyclic saturated hydrocarbon (i.e., —CH₃, —CH₂CH₃, —CH₂CH₂CH₃, —CH(CH₃)₂, —CH₂CH₂CH₂CH₃, —CH₂CH(CH₃)₂, —C(CH₃)₃, etc.).

The term “alkenyl” shall mean a substituting univalent group derived by conceptual removal of one hydrogen atom from a straight or branched-chain acyclic unsaturated hydrocarbon containing at least one double bond (i.e., —CH═CH₂, —CH₂CH═CH₂, —CH═CHCH₃, —CH₂CH═C(CH₃)₂, etc.).

The term “alkynyl” shall mean a substituting univalent group derived by conceptual removal of one hydrogen atom from a straight or branched-chain acyclic unsaturated hydrocarbon containing at least one triple bond (i.e., —C≡CH, —CH₂C≡CH, —C≡CCH₃, —CH₂C≡CCH₂(CH₃)₂, etc.).

The term “alkylene” shall mean a substituting bivalent group derived from a straight or branched-chain acyclic saturated hydrocarbon by conceptual removal of two hydrogen atoms from different carbon atoms (i.e., —CH₂CH₂—, —CH₂CH₂CH₂CH₂—, —CH₂C(CH₃)₂CH₂—, etc.).

The term “alkenylene” shall mean a substituting bivalent group derived from a straight or branched-chain acyclic unsaturated hydrocarbon by conceptual removal of two hydrogen atoms from different carbon atoms (i.e., —CH═CH—, —CH₂CH═CH—, CH₂CH═CHCH₂—, —C(CH₃)═C(CH₃)—, etc.).

The term “cycloalkyl” shall mean a substituting univalent group derived by conceptual removal of one hydrogen atom from a saturated monocyclic hydrocarbon (i.e., cyclopropyl, cyclobutyl, cyclopentyl, cyclohexyl, or cycloheptyl).

The term “heterocycloalkyl” shall mean a substituting univalent group derived by conceptual removal of one hydrogen atom from a heterocycloalkane wherein said heterocycloalkane is derived from the corresponding saturated monocyclic hydrocarbon by replacing one or two carbon atoms with atoms selected from N, O or S. Examples of heterocycloalkyl groups include, but are not limited to, oxiranyl, azetidinyl, pyrrolidinyl, piperidinyl, piperazinyl, and morpholinyl. Heterocycloalkyl substituents can be attached at a carbon atom. If the substituent is a nitrogen containing heterocycloalkyl substituent, it can be attached at the nitrogen atom.

The term “aryl” as used herein refers to a substituting univalent group derived by conceptual removal of one hydrogen atom from a monocyclic or bicyclic aromatic hydrocarbon. Examples of aryl groups are phenyl, indenyl, and naphthyl.

The term “heteroaryl” as used herein refers to a substituting univalent group derived by the conceptual removal of one hydrogen atom from a monocyclic or bicyclic aromatic ring system containing 1, 2, 3, or 4 heteroatoms selected from N, O, or S. Examples of heteroaryl groups include, but are not limited to, pyrrolyl, furyl, thienyl, imidazolyl, pyrazolyl, oxazolyl, isooxazolyl, thiazolyl, pyridyl, pyrimidinyl, pyrazinyl, benzimidazolyl, indolyl, and purinyl. Heteraryl substituents can be attached at a carbon atom or through the heteroatom.

In the ERβ agonist compounds described herein, alkyl, alkenyl, alkynyl, alkylidene, alkenylene, cycloalkyl, cycloalkenyl, heterocycloalkyl, aryl and heteroaryl groups can be further substituted by replacing one or more hydrogen atoms by alternative non-hydrogen groups. These include, but are not limited to, halo, hydroxy, mercapto, amino, carboxy, cyano and carbamoyl.

Whenever the term “alkyl” or “aryl” or either of their prefix roots appear in a name of a substituent (e.g., aryl C₀₋₈ alkyl) it shall be interpreted as including those limitations given above for “alkyl” and “aryl.” Designated numbers of carbon atoms (e.g., C₁₋₁₀) shall refer independently to the number of carbon atoms in an alkyl or cyclic alkyl moiety or to the alkyl portion of a larger substituent in which alkyl appears as its prefix root.

The term “cycloalkylalkyl,” as used herein, shall refer to a system that includes a 3- to 8-membered fully saturated cyclic ring portion and also includes an alkyl portion, wherein cycloalkyl and alkyl are as defined above.

The terms “arylalkyl” and “alkylaryl” include an alkyl portion where alkyl is as defined above and to include an aryl portion where aryl is as defined above. Examples of arylalkyl include, but are not limited to, benzyl, fluorobenzyl, chlorobenzyl, phenylethyl, phenylpropyl, fluorophenylethyl, and chlorophenylethyl. Examples of alkylaryl include, but are not limited to, tolyl, ethylphenyl, and propylphenyl.

The term “heteroarylalkyl,” as used herein, shall refer to a system that includes a heteroaryl portion, where heteroaryl is as defined above, and contains an alkyl portion. Examples of heteroarylalkyl include, but are not limited to, thienylmethyl, thienylethyl, thienylpropyl, pyridylmethyl, pyridylethyl and imidazoylmethyl.

The term “halo” shall include iodo, bromo, chloro and fluoro.

The term “oxy” means an oxygen (O) atom. The term “thio” means a sulfur (S) atom. The term “oxo” means ═O. The term “oximino” means the ═N—O group.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Under standard nonmenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to

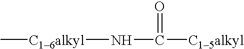

In choosing compounds of the present invention, one of ordinary skill in the art will recognize that the various substituents, i.e. R¹, R², R³, R^{a}, R^{b}, R^{c}, etc. are to be chosen in conformity with well-known principles of chemical structure connectivity.

The ERβ agonist compounds are available in racemic form or as individual enantiomers.

The ERβ agonist compounds of the present invention can be administered in such oral dosage forms as tablets, capsules (each of which includes sustained release or timed release formulations), pills, powders, granules, elixers, tinctures, suspensions, syrups and emulsions. Likewise, they may also be administered in intravenous (bolus or infusion), intraperitoneal, topical (e.g., ocular eyedrop), subcutaneous, intramuscular or transdermal (e.g., patch) form, all using forms well known to those of ordinary skill in the pharmaceutical arts.

The dosage regimen utilizing the ERβ agonist compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the ERβ agonist compounds, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, ERβ agonist compounds may be administered in a single daily dose, or the total daily dosage may be administered in divided doses of two, three or four times daily. Furthermore, preferred ERβ agonist compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittant throughout the dosage regimen.

In the methods of the present invention, the compounds herein described can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

Exemplifying the invention is a pharmaceutical composition comprising an ERβ agonist, an antihypertensive agent and a pharmaceutically acceptable carrier. Also exemplifying the invention is a pharmaceutical composition made by combining an ERβ agonist, an antihypertensive agent and a pharmaceutically acceptable carrier. An illustration of the invention is a process for making a pharmaceutical composition comprising combining an ERβ agonist, an antihypertensive agent and a pharmaceutically acceptable carrier. Further illustrating the invention is the use of an ERβ agonist, an antihypertensive agent and a pharmaceutically acceptable carrier for the preparation of a medicament useful in the treatment of hypertension, cardiac dysfunction or stroke.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The ERβ agonist compounds can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

ERβ agonist compounds may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

According to a further aspect of the present invention, it may be desirable to treat any of the aforementioned conditions with a combination of an ERβ selective agonist and one or more other pharmacologically active agents suitable for the treatment of hypertension, cardiac dysfunction or stroke. The ERβ selective agonist and the other pharmacologically active agent(s) may be administered to a patient simultaneously, sequentially or in combination. For example, the present compound may be employed directly in combination with the other active agent(s), or it may be administered prior, concurrent or subsequent to the administration of the other active agent(s). In general, the currently available dosage forms of the known therapeutic agents for use in such combinations will be suitable.

Calcium channel blocking agents inhibit the movement of ionic calcium across the cell membrane and reduces the force of contraction of muscles of the heart and arteries. Commercially available examples of calcium channel blocking agents include, but are not limited to bepridil (Vascor®), diltiazem (Cardizem®, Cardizem DT®, Cardizem SR®, Dilacor-XR®, Apo-Diltiaz, Nu-Diltiaz, Novo-Diltazem), felodipine (Plendil®, Renedil), isradipine (DynaCirc®), nicardipine (Cardene®), nifedipine (Procardia®, Procardia XL®, Adalat®, Adalat CC®, Adalat PA, Adalat XL, Apo-Nifed, Novo-Nifedin, Nu-Nifed), nimodipine (Nimotop®), verapamil (Calan®, Calan SR®, Isoptin®, Isoptin SR®, Verelan®, Apo-Verap, Novo-Veramil, Nu-Verap) and amlodipine (Norvasc®).

Peripheral vasodilators act by relaxing blood vessels. Examples of peripheral vasodilators include, but are not limited to hydralazine (Apresoline®), isoxuprine (Vasodilan®) and minoxidil (Loniten®).

Beta-adrenergic blocking agents act by reducing adrenergic nerve stimulation, the excitatory nerve stimulation that causes contraction of the muscles in the arteries, veins and heart. Representatives of these agents include beta-adrenergic and alpha/beta adrenergic blockers and examples include, but are not limited to acebutolol (Sectral®), atenolol (Tenormin®, Tenoretic 50®, Tenoretic 100®, Apo-Atenolol), betaxolol (Kerlone®), bisoprolol (Zebeta®, Ziac®), carteolol (Cartrol®), labetalol (Normodyne®, Trandate®), metoprolol (Lopressor®, Lopressor HCT®, Toprol-XL®,), Apo-Metoprolol, Apo-Metoprolol Type L, Betaloc, Betaloc Durules, Novometoprol, Nu-Metop), nadolol (Corgard®, Corzide 4/5®, Corzide 80/5®, Syn-Nadolol), penbutolol (Levatol®), pindolol (Visken®, Novo-Pindol, Syn-Pindolol), propranolol (Inderal®, Inderal LA®, Apo-Propranolol, Detensol, Novopranol, pms Propranolol), sotalol (Betapace®, Sotacor) and timolol (Blocadren®, Apo-Timol, Novo-Timol).

Angiotensin-converting enzyme inhibitors (“ACE inhibitors”) act by inhibiting the production of angiotensin II, a substance that both induces constriction of blood vessels and retention of sodium, which leads to water retension and increased blood volume. Examples of ACE inhibitors include, but are not limited to benazepril (Lotensin®, Lotensin HCT®, Lotrel®), captopril (Capoten®), cilazapril (Inhibace), enalapril (Vasotec®, Vaseretic®), enalaprilat, fosinopril (Aonopril®), lisinopril (Prinivil®, Prinzide®), moexipril (Univasc®), perindopril (Aceon®), quinapril (Accupril®, Accuretic®), ramipril (Altace®) and trandolapril (Mavik® and Tarka®).

Thiazide diuretics act through many mechanisms, including by promoting sodium loss and lowering blood volume. Examples of calcium channel blocking agents include, but are not limited to bendroflumethiazide (Naturetin®), chlorothiazide (Diuril®), chlorthalidone (Hygroton®, Thalitone®, Novo-Thalitone, Apo-Chlorthalidone, Uridon), hydrochlorothiazide (Esidrix®, Rydro-chlor®, Hydro-D®, HydroDIURIL®, Microzide®, Oretic®, Apo-Hydro, Diuchlor, Neo-Codema, Novo-Hydrazide, Urozide), hydroflumethiazde (Diucardin®, Saluron®), methyclothiazide (Aquatensen®, Enduron®, Duretic), metolazone (Diulo®, Mykrox®, Zaroxolyn®), polythiazide(Renese®), quinethazone (Hydromox®) and trichlormethiazide (Metahydrin®, Naqua®D, Trichlorex®).

Angiotensin II receptor antagonists are selective for angiotensin II (type I) receptor and form a newer class of antihypertensive agents. See, Burnier, M, and HR Brunner, (2000), “Angiotensin II receptor antagonists,” Lancet, 355, 637-645. Examples of angiotensin II receptor antagonists include, but are not limited to, losartan, valsartan, irbesartan, candesartan, telmisartan, eprosartan, tasosartan and zolarsartan. Angiotensin II receptor antagonists can be combined with a thiazide diuretic; fixed dosage combinations are available for losartan (Hyzaar®, Cozaar Plus®), valsartan (Diovan HCT(®), irbesartan (Coaprovel®, Karvezide®), candesartan (Atacand HCT®), telmisartan (Micardis HCT®) and eprosartan (Teveten HCT®) with a low dose of hydrochlorothiazide.

Losartan is described in U.S. Pat. Nos. 5,138,069; 5,153,197; 5,210,079; and 5,608,075. It is marketed by Merck & Co., Inc. under the tradenames Cozaar®, Hyzaar® and Cozaar Plus®.

Valsartan is described in U.S. Pat. Nos. 5,399,578 and 6,294,197. It is marketed by Novartis Pharmaceuticals under the tradenames Diovan®, Diovan HCT® and Codiovan®.

Irbesartan is described in U.S. Pat. Nos. 5,270,317 and 6,342,247. It is marketed by Bristol Myers Squibb under the tradenames Avapro®, Avalide®, Coaprovel® and Karvezide®.

Candesartan is described in U.S. Pat. Nos. 5,196,444; 5,534,534; 5,703,110; and 5,705,517. It is marketed under by AstraZeneca under the tradenames Atacand®, and Atacand HCT®.

Telmisartan is described in U.S. Pat. Nos. 5,591,762 and 6,358,986. It is marketed by Boehringer Ingelheim under the tradenames Micardis® and Micardis HCT®.

Eprosartan is described in U.S. Pat. Nos. 5,185,351 and 5,656,650. It is marketed by Bioval Pharmaceuticals, Inc. under the tradenames Teveten® and Teveten HCT®.

### ASSAYS

Estrogen Receptor Binding Assay

The estrogen receptor ligand binding assays are designed as scintillation proximity assays employing the use of tritiated estradiol and recombinant expressed estrogen receptors. The full length recombinant human ER-α, and ER-β proteins are produced in a bacculoviral expression system. ER-α or ER-β extracts are diluted 1:400 in phosphate buffered saline containing 6 mM α-monothiolglycerol. 200 μL aliquots of the diluted receptor preparation are added to each well of a 96-well Flashplate. Plates are covered with Saran Wrap and incubated at 4° C. overnight.

The following morning, a 20 ul aliquot of phosphate buffered saline containing 10% bovine serum albumin is added to each well of the 96 well plate and allowed to incubate at 4° C. for 2 hours. Then the plates are washed with 200 ul of buffer containing 20 mM Tris (pH 7.2), 1 mM EDTA, 10% Glycerol, 50 mM KCl, and 6 mM α-monothiolglycerol. To set up the assay in these receptor coated plates, add 178 ul of the same buffer to each well of the 96 well plate. Then add 20 ul of a 10 nM solution of ³H-estradiol to each well of the plate.

Test compounds are evaluated over a range of concentrations from 0.01 nM to 1000 nM. The test compound stock solutions should be made in 100% DMSO at 100× the final concentration desired for testing in the assay. The amount of DMSO in the test wells of the 96 well plate should not exceed 1%. The final addition to the assay plate is a 2 ul aliquot of the test compound which has been rnade up in 100% DMSO. Seal the plates and allow them to equilibrate at room temperature for 3 hours. Count the plates in a scintillation counter equipped for counting 96 well plates.

Evaluation of a ERβ-Agonist in the Spontaneous Hypertensive Rat (SHR)

Three-week old week old SHR are ovariectomized and allowed ad libitum access to a phytoestrogen-free (PE-) diet containing 8% NaCl. All animals are maintained at constant humidity (65±5%). Temperature (24±1 C), and light/dark cycle (0600-1800, lights on). All procedures related to the use of animals were approved by the Institutional Animal Care and Use Committee at Merck Research Laboratories, West Point, Pa. and conform with the Guide for the Care and Use of Laboratory Animals (US National Institutes of Health, National Research Council, revised 1996). At 7 weeks of age, all rats are anesthetized and instrumented with an implantable arterial pressure transducer/transmitter (TA111PA-C40; Data Sciences). The catheter is inserted into the abdominal aorta (via the femoral artery) iriuediately caudal to the renal arteries with the body of the transmitter sutured to the inside of the anterior abdominal wall. The rats are allowed at least 1 week to recover from the operation and are housed in individual cages throughout the study. Each cage is placed on a receiver panel for recording hemodynamic data via the Dataquest IV software system (Data Sciences).

At 8 weeks of age, the rats are treated (sub-cutaneously, sid for 4 weeks) with either an ER-β agonist, 17-β-estradiol or vehicle (0.1 ml propylene glycol). In vivo measurements include systolic and diastolic blood pressure, urine output, Na/K+ and creatinine excretion, and arterial and venous compliance. After 4 weeks and upon completion of the in vivo measuremaents, the rats are euthanized and the particular tissues are harvested (liver, kidney, uterus) and weighed. The mesenteric artery is also obtained for in vitro studies to determine the contractile response to adrertergic agonists and the relaxation response to nitric oxide releasing agents.

What is claimed is:

1. The use of an ERβ agonist for the preparation of a medicament useful in the treatment of hypertension, cardiac dysfunction or stroke, in a mammal in need thereof.

2. The use according to claim 1 wherein the agonist is a compound of the formula: 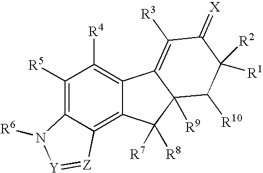
wherein X is O or N—OR^{a};
Y is N or CH;
Z is N or CR^{f};
R¹ is hydrogen or C₁₋₆alkyl;
R² is hydrogen, hydroxy, iodo or C₁₋₆alkyl;
R³ is hydrogen, fluoro, chloro, bromo, iodo, cyano, nitro, NR^{a}R^{c}, OR^{a}, S(O)R^{a}, SO₂R^{a}, SR^{a}, C(═O)R^{a}, CO₂R^{C}, CONR^{a}R^{c}, C₁₋₁₀alkyl, C₂₋₁₀alkenyl, C₂₋₁₀alkynyl, C₃₋₇cycloalkyl, 4-7 membered heterocycloalkyl, cycloalkylalkyl, aryl, heteroaryl, arylalkyl, or heteroarylalkyl, wherein said alkyl, alkenyl, alkynyl, cycloalkyl, aryl and heteroaryl groups are optionally substituted with 1, 2 or 3 groups selected from the group consisting of fluoro, chloro, bromo, iodo, cyano, OR^{a}, NR^{a}R^{c}, O(C═O)R^{a}, O(C═O)NR^{a}R^{c}, NR^{a}(C═O)R^{c}, NR^{a}(C═O)OR^{c}, C(═O)R^{a}, CO₂R^{a}, CONR^{a}R^{c}, CSNR^{a}R^{c}, SR^{a}, S(O)R^{a}, SO₂R^{a}, SO₂NR^{a}R^{c}, LR^{d}, and MLR^{d};
R⁴ is hydrogen, hydroxy, methyl, fluoro or chloro;
R⁵ is hydrogen, hydroxy, fluoro or chloro;
R⁶ is hydrogen, (C═O)R^{a} or (C═O)OR^{a};
R⁷ is hydrogen, fluoro, chloro or C₁₋₆alkyl;
R⁸ is hydrogen, fluoro, chloro or C₁₋₆alkyl;
or R⁷ and R⁸, when taken together with the carbon atom to which they are attached, form a carbonyl group;
R⁹ is hydrogen, C₁₋₁₀alkyl, C₂₋₁₀alkenyl, C₃₋₆cycloalkyl, C₃₋₆cycloalkylalkyl, aryl, heteroaryl, arylalkyl or heteroarylalkyl, wherein said alkyl, alkenyl, cycloalkyl, cycloalkylalkyl, aryl, heteroaryl, arylalkyl and heteroarylalkyl groups are optionally substituted with chloro, bromo, OR^{b}, SR^{b} or 1-5 fluoro;
or R⁹ and R¹, when taken together with the three intervening carbon atoms to which they are attached, form a 5-6 membered cycloalkyl ring which is optionally substituted with 1-3 fluoro, chloro, C₁₋₆alkyl, C₂₋₆alkenyl or C₃₋₆cycloalkylalkyl, wherein said alkyl, alkenyl and cycloalkylalkyl groups are optionally substituted with chloro, OR^{b}, SR^{b} or 1-5 fluoro;
R¹⁰ is hydrogen or C₁₋₁₀alkyl;
R^{a} is hydrogen, C₁₋₁₀alkyl or phenyl, wherein said alkyl group is optionally substituted with hydroxy, amino, O(C₁₋₄alkyl), NH(C₁₋₄alkyl), N(C₁₋₄alkyl)₂, phenyl or 1-5 fluoro, and wherein said phenyl group is optionally substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H, and C(O)(C₁₋₄alkyl);
R^{b} is hydrogen, C₁₋₁₀alkyl, benzyl or phenyl, wherein said phenyl group is optionally substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H and C(O)(C₁₋₄alkyl);
R^{c} is hydrogen, C₁₋₁₀alkyl or phenyl, wherein said phenyl group is optionally substituted with 1-3 substituents independently selected from the group consisting of C₁₋₄alkyl, OH, O(C₁₋₄alkyl), NH₂, NH(C₁₋₄alkyl), NH(C₁₋₄alkyl)₂, halo, CN, NO₂, CO₂H, CO₂(C₁₋₄alkyl), C(O)H and C(O)(C₁₋₄alkyl);
or R^{a} and R^{c}, whether or not on the same atom, can be taken together with any attached and intervening atoms to form a 4-7 membered ring;
R^{d} is NR^{b}R^{c}, OR^{a}, CO₂R^{a}, O(C═O)R^{a}, CN, NR^{c}(C═O)R^{b}, CONR^{a}R^{c}, SO₂NR^{a}R^{c} or a 4-7 membered N-heterocycloalkyl ring that is optionally interrupted by O, S, NR^{c}, or C═O;
R^{e} is hydrogen, C₁₋₆alkyl, C₂₋₆alkenyl, CF₃, halo, O(C₁₋₄alkyl), NH₂, NH(Cl 4alkyl) or N(C₁₋₄alkyl)₂;
R^{f} is hydrogen, C₁₋₆alkyl, C₂₋₆alkenyl, CF₃, halo, O(C₁₋₄alkyl), NO₂, NH₂, NH(C₁₋₄alkyl) or N(C₁₋₄alkyl)₂;
L is CR^{b}R^{c}, C₂₋₆ alkylene or C₂₋₆ alkenylene, wherein said alkylene and alkenylene groups are optionally interrupted by O, S, or NR^{c};
M is O, S, NR^{c}, C═O, O(C═O), (C═O)O, NR^{c}(C═O) or (C═O)NR^{c}; or a salt or stereoisomer thereof.

3. The use according to claim 2 wherein
X is O, N—OH or N—OCH₃;
Y is N or CH;
Z is N, CH, CF or CCl;
R¹ is hydrogen or C₁₋₃alkyl;
R² is hydrogen, hydroxy, iodo or C₁₋₃alkyl;
R³ is hydrogen, chloro, bromo, iodo, C₁₋₁₀alkyl, C₂₋₁₀alkenyl, C₃₋₇cycloalkyl or aryl, wherein said alkyl, alkenyl, cycloalkyl and aryl groups are optionally substituted with 1, 2 or 3 groups selected from the group consisting of fluoro, OR^{a}, NR^{a}R^{c}, LR^{d} and MLR^{d};
R⁴ is hydrogen, methyl or fluoro;
R⁵ is hydrogen or fluoro;
R⁶ is hydrogen or C(═O)OR^{a};
R⁷ is hydrogen or C₁₋₆alkyl;
R⁸ is hydrogen or C₁₋₆alkyl;
R⁹ is C₁₋₁₀alkyl, C₂₋₁₀alkenyl, C₃₋₆cycloalkyl or C₃₋₆ cycloalkylalkyl;
R¹⁰ is hydrogen.

4. The use according to claim 2 wherein
X is O and Z is N or CH.

5. The use according to claim 1 wherein the agonist is:
9a-ethyl-1,6-dimethyl-8,9,9a,10-tetrahydroindeno[2,1-e]indol-7(3H)-one;
9a-ethyl-6-methyl-8,9,9a,10-tetrahydroindeno[2,1-e]indol-7(3H)-one;
1-chloro-9a-ethyl-6-methyl-8,9,9a,10-tetrahydroindeno[2,1-e]indol-7(3H)-one;
9a-ethyl-6-methyl-1-nitro-8,9,9a,10-tetrahydroindeno[2,1-e]indol-7(3H)-one;
6-acetyl-9a-butyl-4-fluoro-8,9,9a,10-tetrahydroindeno[2,1-e]indol-7(3H)-one;
6-methyl-9a-propyl-8,9,9a,10-tetrahydroindeno[2,1-e]indol-7(3H)-one;
9a-ethyl-4-fluoro-6-methyl-8,9,9a,10-tetrahydroindeno[2,1-e]indol-7(3H)-one;
6,9a-diethyl-4-fluoro-8,9,9a,10-tetrahydroindeno[2,1-e]indol-7(3H)-one;
9a-butyl-4-fluoro-6-methyl-8,9,9a,10-tetrahydroindeno[2,1-e]indol-7(3H)-one;
9a-butyl-6-ethyl-4-fluoro-8,9,9a,10-tetrahydroindeno[2,1-e]indol-7(3H)-one;
6,9a-dimethyl-8,9,9a,10-tetrahydroindeno[2,1-e]indazol-7(3H)-one;
6-bromo-9a-methyl-8,9,9a,10-tetrahydroindeno[2,1-e]indazol-7(3,H)-one;
9a-ethyl-8,9,9a,10-tetrahydroindeno[2,1-e]indazol-7(3H)-one;
9a-ethyl-6-methyl-8,9,9a,10-tetrahydroindeno[2,1-e]indazol-7(3H)-one;
6-bromo-9a-ethyl-8,9,9a,10-tetrahydroindeno[2,1-e]indazol-7(3H)-one;
9a-ethyl-6-trifluoromethyl-8,9,9a,10-tetrahydroindeno[2,1-e]indazol-7(3H)-one;
9a-ethyl-6-{4-[2-(1-piperidinyl)ethoxy]phenyl}-8,9,9a,10-tetrahydroindeno[2,1-e]indazol-7(3H)-one hydrochloride salt;
9a-ethyl-6-(4-hydroxyphenyl)-8,9,9a,10-tetrahydroindeno[2,1-e]indazol-7(3H)-one;
9a-ethyl-6-vinyl-8,9,9a,10-tetrahydroindeno[2,1-e]indazol-7(3H)-one;
6,9a-diethyl-8,9,9a,10-tetrahydroindeno[2,1-e]indazol-7(31)-one;
6-allyl-9a-ethyl-8,9,9a,10-tetrahydroindeno[2,1-e]indazol-7(3H)-one;
9a-ethyl-6-isopropyl-8,9,9a,10-tetrahydroindeno[2,1-e]indazol-7(3H)-one;
6-butyl-9a-ethyl-8,9,9a,10-tetrahydroindeno[2,1-e]indazol-7(3H)-one;
6-cyclopentyl-9a-ethyl-8,9,9a,10-tetrahydroindeno[2,1-e]indazol-7(3H)-one;
6-cyano-9a-ethyl-8,9,9a,l0-tetrahydroindeno[2,1-e]indazol-7(3H)-one;
9a-ethyl-6-methoxy-8,9,9a,10-tetrahydroindeno[2,1-e]indazol-7(3H)-one;
1-chloro-9a-ethyl-6-methyl-8,9,9a,10-tetrahydroindeno[2,1-e]indazol-7(3H)-one;
1-bromo-9a-ethyl-6-methyl-8,9,9a,10-tetrahydroindeno[2,1-e]indazol-7(3H)-one;
9a-ethyl-6-methyl-9,9a-dihydroindeno[2,1-e]indazole-7,10(3H,8H)-dione;
10-chloro-9a-ethyl-6-methyl-8,9,9a,10-tetrahydroindeno[2,1-e]indazol-7(3H)-one;
10-azido-9a-ethyl-6-methyl-8,9,9a,10-tetrahydroindeno[2,1-e]indazol-7(3H)-one;
6-bromo-9a-ethyl-9,9a-dihydroindeno[2,1-e]indazole-7,10(3H,8H)-dione;
10-amino-9a-ethyl-6-methyl-8,9,9a,10-tetrahydroindeno[2,1-e]indazol-7(3H)-one;
9a-ethyl-10-methoxy-6-methyl-8,9,9a,10-tetrahydroindeno [2,1-e]indazol-7(3H)-one;
9a-ethyl-6,10-dimethyl-8,9,9a,10-tetrahydroindeno[2,1-e]indazol-7(3H)-one;
9a-ethyl-4-fluoro-6-methyl-8,9,9a,10-tetrahydroindeno[2,1-e]indazol-7(3H)-one;
6,9a-diethyl-4-fluoro-8,9,9a,10-tetrahydroindeno[2,1-e]indazol-7(3H)-one;
6-bromo-9a-ethyl-4-fluoro-8,9,9a,10-tetrahydroindeno[2,1-e]indazol-7(3H)-one;
9a-ethyl-4-fluoro-6-trifluoromethyl-8,9,9a,10-tetrahydroindeno[2,1-e]indazol-7(3H)-one;
6-methyl-9a-propyl-8,9,9a,10-tetrahydroindeno[2,1-e]indazol-7(3H)-one;
6-bromo-9a-propyl-8,9,9a,10-tetrahydroindeno[2,1-e]indazol-7(3H)-one;
6-cyano-9a-propyl-8,9,9a,10-tetrahydroindeno[2,1-e]indazol-7(3H)-one;
6-methyl-9a-propyl-8,9,9a,10-tetrahydroindeno[2,1-e]indazol-7(3H)-one oxime;
9a-butyl-8,9,9a,10-tetrahydroindeno[2,1-e]indazol-7(3H)-one;
6-bromo-9a-butyl-8,9,9a,10-tetrahydroindeno[2,1-e]indazol-7(3H)-one;
9a-butyl-6-trifluoromethyl-8,9,9a,10-tetrahydroindeno[2,1-e]indazol-7(3H)-one;
9a-butyl-6-methyl-8,9,9a,10-tetrahydroindeno[2,1-e]indazol-7(3H)-one;
9a-butyl-6-ethyl-8,9,9a,10-tetrahydroindeno[2,1-e]indazol-7(3H)-one;
9a-(3,3-dimethylbutyl)-6-methyl-8,9,9a,10-tetrahydroindeno[2,1-e]indazol-7(3H)-one;
9a-butyl-6-ethyl4-fluoro-8,9,9a,10-tetrahydroindeno[2,1-e]indazol-7(3H)-one;
6-acetyl-9a-butyl-4-fluoro-8,9,9a,10-tetrahydroindeno[2,1-e]indazol-7(3H)-one;
9a-butyl-4-fluoro-6-methyl-8,9,9a, 10-tetrahydroindeno[2,1-e]indazol-7(3H)-one;
6-bromo-9a-butyl4-fluoro-8,9,9a,10-tetrahydroindeno[2,1-e]indazol-7(3H)-one;
9a-butyl-6-cyano-4-fluoro-8,9,9a,10-tetrahydroindeno[2,l -e]indazol-7(3H)-one;
9a-butyl-4-fluoro-6-trifluoromethyl-8,9,9a,10-tetrahydroindeno[2,1-e]indazol-7(3H)-one;
6-methyl-3,9,10,11-tetrahydro-8,10a-methanoazuleno[2,1-e]indazol-7(8H)-one;
6-ethyl-3,9,10,11-tetrahydro-8,10a-methanoazuleno[2,1-e]indazol-7(8H)-one;
9a-ethyl-6-methyl-8,9,9a,10-tetrahydrofluoreno[1,2-d]imidazol-7(3H)-one;
6-bromo-9a-ethyl-8,9,9a,10-tetrahydrofluoreno[1,2-d]imidazol-7(3H)-one;
6,9a-diethyl-4-fluoro-8,9,9a,10-tetrahydrofluoreno[1,2-d]imidazol-7(3H)-one;
9a-butyl-6-ethyl-4-fluoro-8,9,9a,10-tetrahydrofluoreno[1,2-d]imidazol-7(3H)-one;
9a-ethyl-8,9,9a,10-tetrahydrofluoreno[1,2-d][1,2,3]triazol-7(3H)-one;
9a-ethyl-6-methyl-8,9,9a,10-tetrahydrofluoreno[1,2-d][1,2,3]triazol-7(3H)-one;
6-allyl-9a-ethyl-8,9,9a,10-tetrahydrofluoreno[1,2-d][1,2,3 ]triazol-7(3H)-one;
9a-ethyl-6-propyl-8,9,9a,10-tetrahydrofluoreno[1,2-d][1,2,3]triazol-7(3H)-one;
9a-ethyl-6-trifluoromethyl-8,9,9a,10-tetrahydrofluoreno[1,2-d][1,2,3]triazol-7(3H)-one;
6-bromo-9a-ethyl-8,9,9a,10-tetrahydrofluoreno[1,2-d][1,2,3]triazol-7(3H)-one;
6,9a-diethyl-8,9,9a,10-tetrahydrofluoreno[1,2-d][1,2,3]triazol-7(3H)-one;
6-butyl-9a-ethyl-8,9,9a,10-tetrahydrofluoreno[1,2-d][1,2,3]triazol-7(3H)-one;
9a-ethyl-6-(4-hydroxyphenyl)-8,9,9a,10-tetrahydrofluoreno[1,2-d][1,2,3]triazol-7(3H)-one;
6-bromo-9a-propyl-8,9,9a,10-tetrahydrofluoreno[1,2-d][1,2,3]triazol-7(3H)-one;
6-methyl-9a-propyl-8,9,9a,10-tetrahydrofluoreno[1,2-d][1,2,3]triazol-7(3H)-one;
9a-propyl-6-vinyl-8,9,9a,10-tetrahydrofluoreno[1,2-d][1,2,3]triazol-7(3H)-one;
6-ethyl-9a-propyl-8,9,9a,10-tetrahydrofluoreno[1,2-d][1,2,3]triazol-7(3H)-one;
6-allyl-9a-propyl-8,9,9a,10-tetrahydrofluoreno[1,2-d][1,2,3]triazol-7(3H)-one;
6,9a-dipropyl-8,9,9a,10-tetrahydrofluoreno[1,2-d][1,2,3]triazol-7(3H)-one;
6-bromo-9a-butyl-8,9,9a,10-tetrahydrofluoreno[1,2-cl][1,2,3]triazol-7(3H) -one;
9a-butyl-6-methyl-8,9,9a,10-tetrahydrofluoreno[1,2-d][1,2,3]triazol-7(3H)-one;
9a-butyl-6-ethyl-8,9,9a,10-tetrahydrofluoreno[1,2-d][1,2,3]triazol-7(3H)-one;
6-allyl-9a-butyl-8,9,9a,10-tetrahydrofluoreno[1,2-d][1,2,3]triazol-7(3H)-one;
9a-butyl-6-propyl-8,9,9a,10-tetrahydrofluoreno[1,2-d][1,2,3]triazol-7(3H)-one;
9a-butyl-6-trifluoromethyl-8,9,9a,10-tetrahydrofluoreno[1,2-d][1,2,3]triazol-7(3H)-one;
9a-butyl-6-(2-furyl)-8,9,9a,10-tetrahydrofluoreno[1,2-d][1,2,3]triazol-7(3H)-one;
6,9a-diethyl-4-fluoro-8,9,9a,10-tetrahydrofluoreno[1,2-d][1,2,3]triazol-7(3H)-one;
9a-butyl-6-ethyl-4-fluoro-8,9,9a,10-tetrahydrofluoreno[1,2-d][1,2,3]triazol-7(3H)-one;
or a salt or stereoisomer thereof.

6. The use according to claim 1 which further comprises an antihypertensive agent selected from the group consisting of a calcium channel blocking agent, a beta-adrenergic blocking agent, an angiotensin-converting enzyme inhibitor, angiotensin-II receptor antagonist, a thiazide diuretic and a peripheral vasodilator.

7. The use according to claim 6 wherein the calcium channel blocking agent is bepridil, diltiazem, felodipine, isradipine, nicardipine, nifedipine, nimodipine, verapamil or amlodipine.

8. The use according to claim 6 wherein the beta-adrenergic blocking agent is acebutolol, atenolol, betaxolol, bisoprolol, carteolol, labetalol, metoprolol, nadolol, penbutolol, pindolol, propranolol, sotalol or timolol.

9. The use according to claim 6 wherein the angiotensin-converting enzyme inhibitor is benazepril, captopril, cilazapril, enalapril, enalaprilat, fosinopril, lisinopril, moexipril, perindopril, quinapril, ramipril or trandolapril.

10. The use according to claim 6 wherein the angiotensin-II receptor antagonist is losartan, valsartan, irbesartan, candesartan, telmisartan or eprosartan.

11. The use according to claim 10 wherein the angiotensin-II receptor antagonist is losartan.

12. The use according to claim 6 wherein the thiazide diuretic is bendroflumethiazide, chlorothiazide, chlorthalidone, hydrochlorothiazide, hydroflumethiazde, methyclothiazide, metolazone, polythiazide, quinethazone or trichlormethiazide.

13. The use according to claim 6 wherein the peripheral vasodilator is hydralazine, isoxuprine or minoxidil.

14. A pharmaceutical composition comprising an ERβ agonist, an antihypertensive agent and a pharmaceutically acceptable carrier.
